(19) United States
(12) Patent Application Publication
(10) Pub. No.: US 2007/0066867 A1
(43) Pub. Date: Mar. 22, 2007

(54) Bronchoscopic application of therapeutic agent to upper airway and nasal tissue

(75) Inventor: Farhan Taghizadeh, Albuquerque, NM (US)

Correspondence Address: MCDERMOTT WILL & EMERY LLP, 18191 VON KARMAN AVE., SUITE 500, IRVINE, CA, 92612-7108, US

(73) Assignee: University Of Rochester, Rochester, NY (US)

(21) Appl. No.: 11/488,394

(22) Filed: Jul. 18, 2006

Related U.S. Application Data
(60) Provisional application No. 60/700,563, filed on Jul. 19, 2005.

(51) Int. Cl. A61B 1/00

(52) U.S. Cl. 600/104; 600/153

(57) ABSTRACT

Described herein are devices and methods for facilitating the application of therapeutic agents, such as silver nitrate, in the nasal cavity, for example, to achieve hemostasis for epistaxis, and in the airway, for example, to reduce the size of granulation tissue. In some embodiments, the methods and devices utilize a fiberoptic laryngoscope or flexible bronchoscope, which can be used for the examination and treatment of the upper airway.

### RELATED APPLICATION

This application claims priority benefit of U.S. Provisional Application No. 60/700,563, filed Jul. 19, 2005, the entire contents of which are incorporated herein by reference.

### BACKGROUND OF THE INVENTIONS

### DESCRIPTION OF THE RELATED ART

Epistaxis, or nasal bleeding, is a major health issue that impacts a larger segment of the population as people age and the use of anticoagulant drugs increases. Anticoagulants increase the likelihood of epistaxis and make it more difficult to stop the bleeding. Another health issue that plagues a segment of the population is the presence of granulation tissue in the upper airway. These granulations can cause airway obstruction and are often difficult to reach without the patient being under anesthesia.

One technique for epistaxis control and shrinkage of airway granulation tissue is the application of a caustic agent, such as silver nitrate (AgNO3). This technique is employed to achieve hemostasis and to get granulation tissue in the airway to reduce in size. Silver nitrate is commonly prepared on the end of sticks, with the substance having the ability to cauterize bleeding and help reduce the size of hypertrophic mucosa and granulation tissue in the airway.

### SUMMARY OF THE INVENTIONS

Silver nitrate applications to the anterior nose are performed and are not necessarily complex procedures. However, application of silver nitrate into the posterior nose or nasal cavity can be difficult, particularly if the health care professional is using an endoscope. Additionally, applying silver nitrate into the airway presents several issues. Silver nitrate can be applied to the airway, but such application is presently accomplished with limited control of the silver nitrate.

Disclosed herein are devices and methods for facilitating the application of therapeutic agents, such as silver nitrate, in the nasal cavity to achieve hemostasis for epistaxis and in the airway to reduce the size of granulation tissue. The devices utilize a fiberoptic laryngoscope or flexible bronchoscope, which can be used for the examination of the upper airway. The devices may utilize other types of devices. For example, a rigid bronchoscope for evaluating and conducting procedures on the upper airway may also be utilized in connection with the disclosure and embodiments provided herein.

In one application, a method is disclosed for treating tissues through scopes, such as those described above. The method contemplates the use of operating members, such as wires, that can be passed through the proximal end of the bronchoscope and brought out at the distal tip. These wires can then be observed in the field of view at the tip of the bronchoscope.

In one embodiment, a novel method for applying therapeutic agents, such as silver nitrate, directly through a nasal or bronchial endoscope is disclosed. The method contemplates using a flexible wire apparatus or using a separate rigid apparatus connected to the wire. In other embodiments, methods and devices for placing silver nitrate on the ends of these wires are disclosed. The apparatus are configured such that the silver nitrate can be brought into the airway through a rigid or flexible bronchoscope to cauterize hypertrophic mucosa and granulation tissue in the airway. In yet another embodiment, a rigid nasal endoscope is disclosed that is configured to allow the passage of these wires to control epistaxis, and a new rigid tube to separately apply silver nitrate to the posterior nose.

In one embodiment, an endoscopic device is disclosed, the device including a protective cover configured that is positionable in an open configuration and a closed configuration, the protective cover being sized and shaped for insertion through a lumen in an endoscope. The device also includes an applicator tip configured to apply a therapeutic agent to a tissue, the applicator tip being configured to be at least partially covered by the protective cover when the protective cover is in a closed configuration, and the applicator tip further being configured to be exposed when the protective cover is in an open configuration. The device also includes an actuator that is configured to move the protective cover between the open configuration and the closed configuration.

In another embodiment, an endoscopic therapeutic device is disclosed comprising an endoscope configured for viewing of a patient, the endoscope having a lumen therethrough for insertion of an operating instrument and an operating member having a proximal end and a distal end. The distal end of the operating member preferably comprises a protective covering that is configured to enclose therein a therapeutic agent, and the proximal end of the operating member is configured to be connected to an actuator. The actuator is preferably configured to activate movement of the protective covering and expose the caustic agent, and the operating member is preferably configured for insertion through the lumen of the endoscope.

Other embodiments are also disclosed herein, and the above-described summary is not intended to limit the embodiments disclosed below or recited in the claims.

### BRIEF DESCRIPTION OF THE DRAWINGS

These and other features and advantages of the disclosure, features, aspects, and embodiments will become more apparent upon reading the following detailed description and with reference to the accompanying drawings of various embodiments that exemplifies the inventions. The drawings comprise 17 figures.

FIG. 1A is an illustration of an endoscope extending through the nasal passage of a patient into the left main bronchi.

FIG. 1B depicts an endoscopic forcep that is operated at a proximal end by a button.

FIG. 2A depicts a flexible endoscope with an endoscopic caustic agent application device about to be inserted into a side port of the endoscope.

FIG. 2B depicts a flexible endoscope with an endoscopic caustic agent application device extending through a distal opening in the endoscope.

FIG. 3 illustrates a rigid endoscope with an endoscopic caustic agent application device extending from a distal portion thereof.

FIG. 4 is an illustration of a caustic agent application base that is used in connection with the endoscopic caustic agent application device.

FIG. 5A is a schematic illustration of an embodiment of an endoscopic caustic agent application device in a closed position.

FIG. 5B is a schematic illustration of an embodiment of the application device of FIG. 5A in a partially open position.

FIG. 5C is a schematic illustration of an embodiment of the application device of FIG. 5A in an open position and prepared for application of the caustic agent.

FIG. 6 illustrates an embodiment of an endoscopic caustic agent application device in accordance with principles disclosed herein.

FIGS. 7A-C illustrate another embodiment of an endoscopic caustic agent application device.

FIG. 8 schematically illustrates a rigid endoscope and a flexible endoscopic caustic agent application device inserted through a side port and extending from a distal portion of the endoscope.

FIG. 9 schematically illustrates a rigid endoscope and a rigid endoscopic caustic agent application device to be used therewith.

FIG. 10 depicts the application of the caustic agent using the endoscopic caustic agent application device as viewed through the endoscope.

### DETAILED DESCRIPTION OF THE PREFERRED EMBODIMENTS

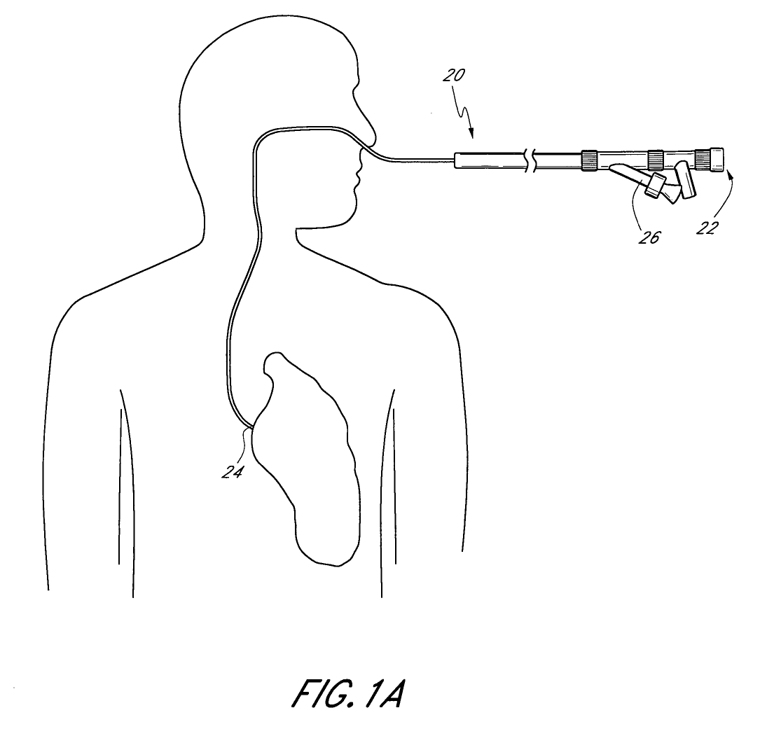

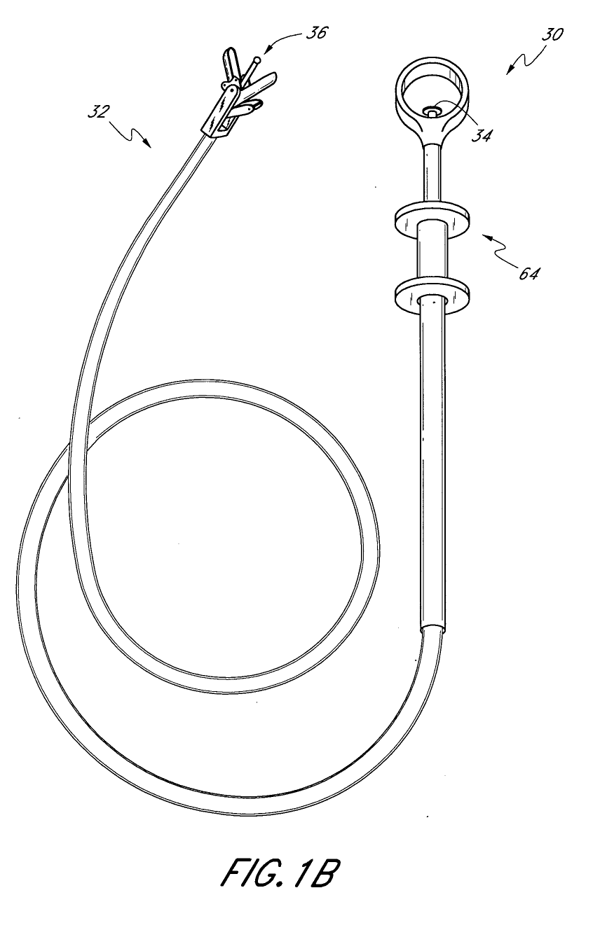

FIG. 1A illustrates a flexible endoscope, or bronchoscope 20, extending through a patient's nasal cavity, trachea, and into the left main bronchi. The bronchoscope 20 is used for viewing a patient's airway passages for diagnostic and therapeutic purposes. Through a bronchoscope 20, a practitioner may observe growths or lesions existing in the sinuses, trachea, or lungs.

The bronchoscope 20 has a proximal end 22, through which the practitioner looks, or to which electronic equipment is attached, for viewing the portion of the airway at which a distal end 24 is located. A proximal port 26 is located near or at the proximal end 22 for passage of instruments through the bronchoscope 20. The proximal port 26 is in communication with a lumen that extends through the bronchoscope 20 to a position adjacent or at the distal end 24.

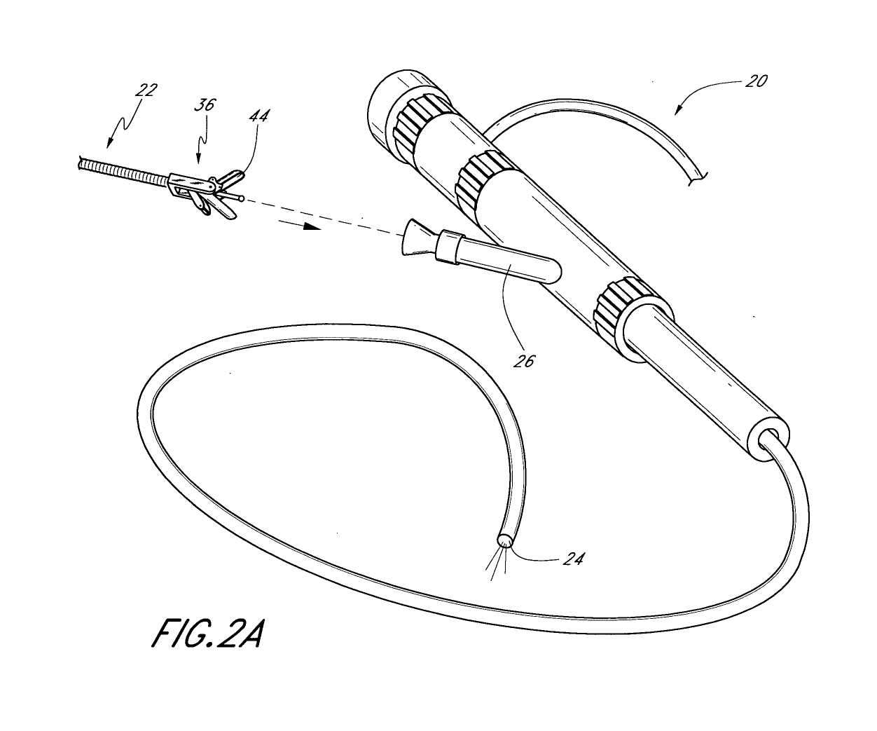

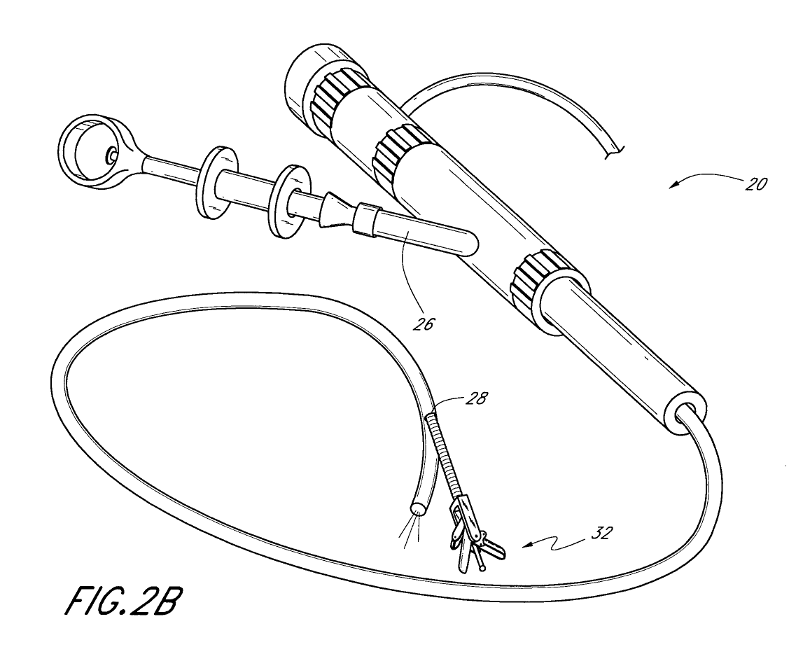

At the distal end 24, the bronchoscope 20 has a distal port 28 (FIG. 2B) that is also in communication with the lumen and which provides communication between the lumen and the exterior of the bronchoscope 20. The proximal port 26, the lumen, and the distal port 28 cooperate to allow the practitioner to insert an instrument therethrough for therapeutic procedures within the patient.

For example, an operating wire 30, depicted in FIG. 1B, may be inserted in the proximal port 26 and fed through the lumen. An operating end 32 is preferably disposed on a distal portion of the wire 30 for manipulating or treating structures within the patient. The wire 30 preferably includes a button 34, or other operating mechanism, on the proximal portion thereof for the practitioner to manipulate the operating end 32 of the wire 30. As depicted in FIG. 1B, the operating end 32 preferably includes a distal tip 36 that is configured to apply caustic agents to portions of the patient's airway.

FIG. 2A depicts a flexible bronchoscope, laryngoscope, or other endoscope 20. The scope 20 is depicted with a proximal port 26, through which the operating end 32 of an operating wire 30 is to be inserted. FIG. 2B depicts the operating end 32 of the operating wire 30 extending from the distal port 28 of the scope 20. This configuration permits treatment of a portion of the patient's airway within the line of sight of the scope 20.

The scopes 20 used in the treatment of a patient described herein may be a flexible scope, as depicted in FIGS. 1A, 2A, and 2B. When a flexible scope is used, a flexible operating wire 30 can also be used to permit the flexible features of the scope to be utilized. In some applications, a rigid wire can also be used. In many procedures, it may be desirable to use a rigid scope 20, as depicted in FIG. 3. When a rigid scope 20 is used, a flexible or a rigid operating wire 30 can be used. For example, it may be desirous to use a rigid scope 20 for diagnostic and/or therapeutic treatment of the sinuses, such as in the case of epistaxis or endoscopic sinus surgery. For epistaxis, it may be desirable to utilize a rigid scope 20 to locate and treat a lesion within the nose or sinus that is bleeding. For endoscopic sinus surgery, it may be desirous for removing blockages in the sinuses to treat sinusitis, which may cause the sinuses to swell, become clogged, cause pain, and/or impair breathing.

Silver nitrate may be used in the treatment of epistaxis or granulations within the airway. Silver nitrate is a caustic agent that may be used to stop the flow of blood from a lesion or to reduce the size of granulations. Other caustic agents may also be used. For example, other caustic agents that may be used include, but are not limited to, zinc chloride, copper sulfate, phenol, acids, alkali, iodine, absolute alcohol, potassium permanganate, formalin or combinations thereof. Furthermore, depending on the intended use, the concentration of the selected caustic agent is routinely adjusted. Where deeper penetration of the caustic agent is preferred, a more concentrated solution of the caustic agent should be used. Other characteristics such as speed and severity of cautery are adjusted as well, depending on the desired use and may be achieved by altering viscosity.

The activity of the caustic agent can be readily controlled by using silver compounds such as silver nitrate and silver thiocyanate or other compounds which can release silver ions. The silver ions react with the sulfides, proteins, and chlorides in cells. Since the sulfides and chlorides are vital to cell metabolism, the reaction results in necrosis of the cells. Another potentially useful agent is iodine, which is radiopaque like silver. Compositions containing iodine react with the target tissue as the result of the release of elemental free iodine and the reaction can be stopped by forming a stable compound, for example, sodium iodide by instilling sodium chloride. In one embodiment, silver nitrate and DEXTRAN 70® are utilized together because they are easy to work with, are controllable, and are recognized by the medical profession and government regulatory agencies as acceptable agents for human use.

The above-description regarding various caustic agents is merely exemplary and is not intended to limit the type, application, or operation of the caustic agent. Applications of caustic agents are further described in U.S. patent application Ser. No. 09/882,811, filed Jun. 18, 2001, entitled, “Liquid Cautery Catheter,” and U.S. Pat. No. 6,197,351, filed Sep. 17, 1997, entitled, “Intrauterine Chemical Necrosing Method and Composition,” the entirety of both of which are incorporated herein by reference and are made a part of this specification.

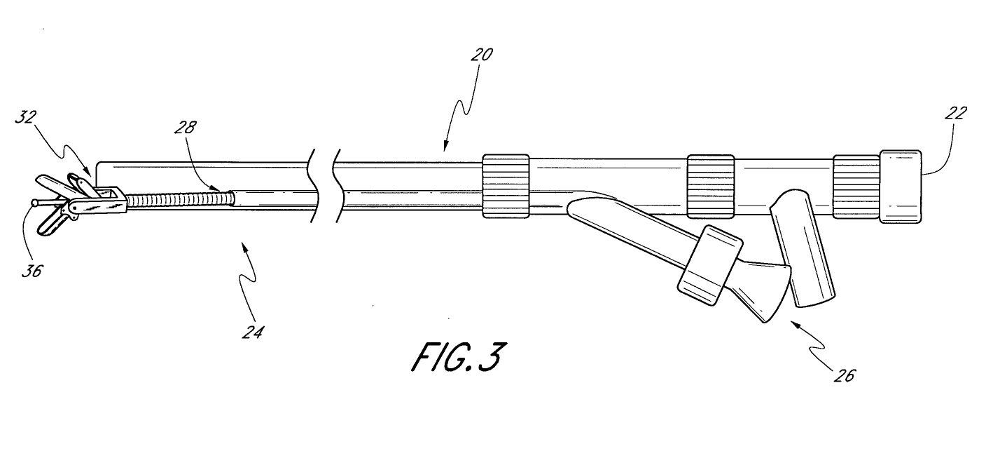

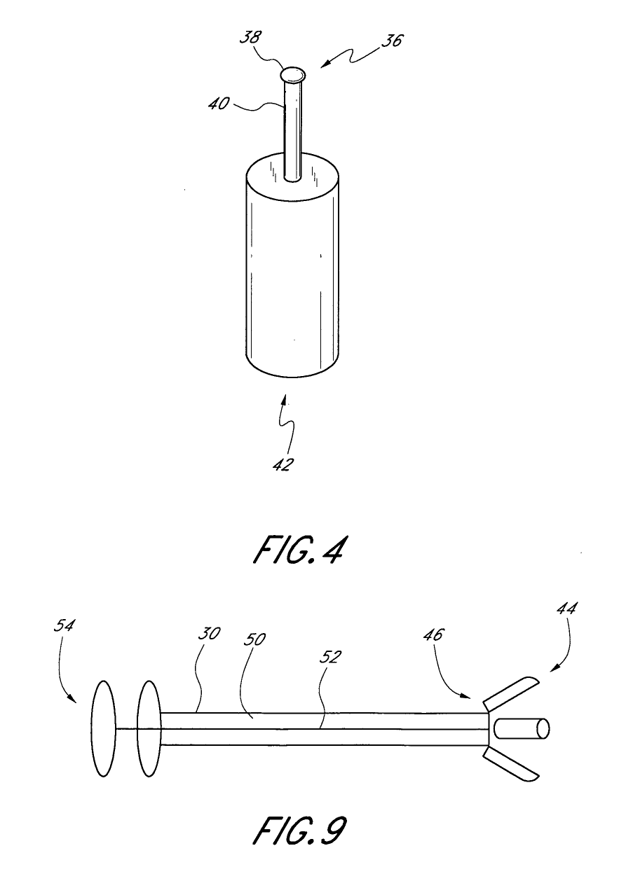

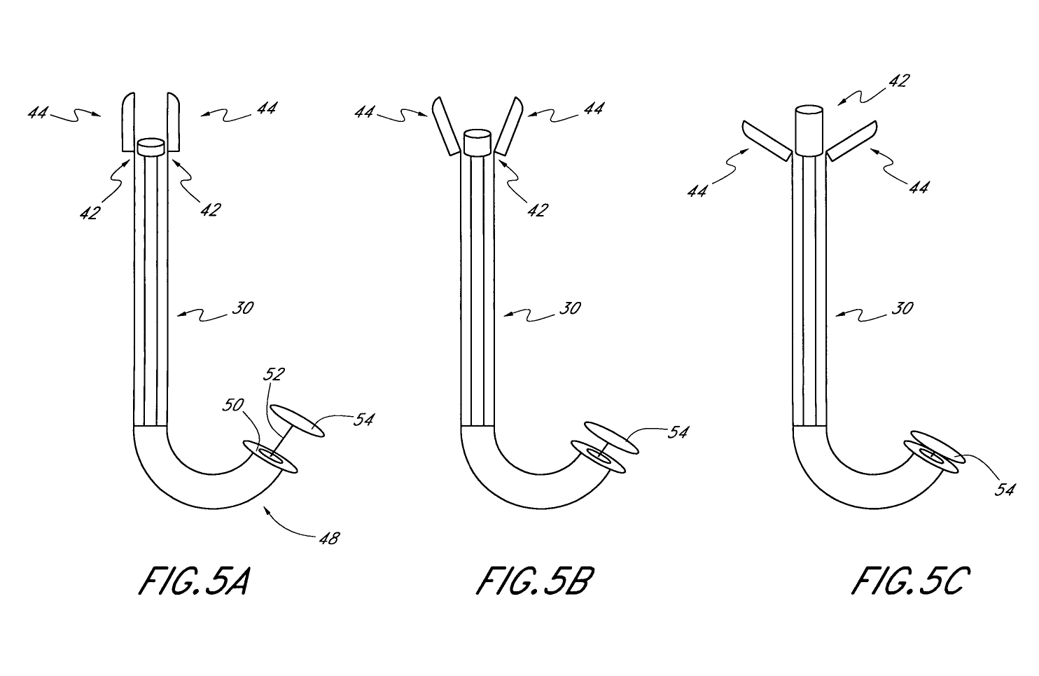

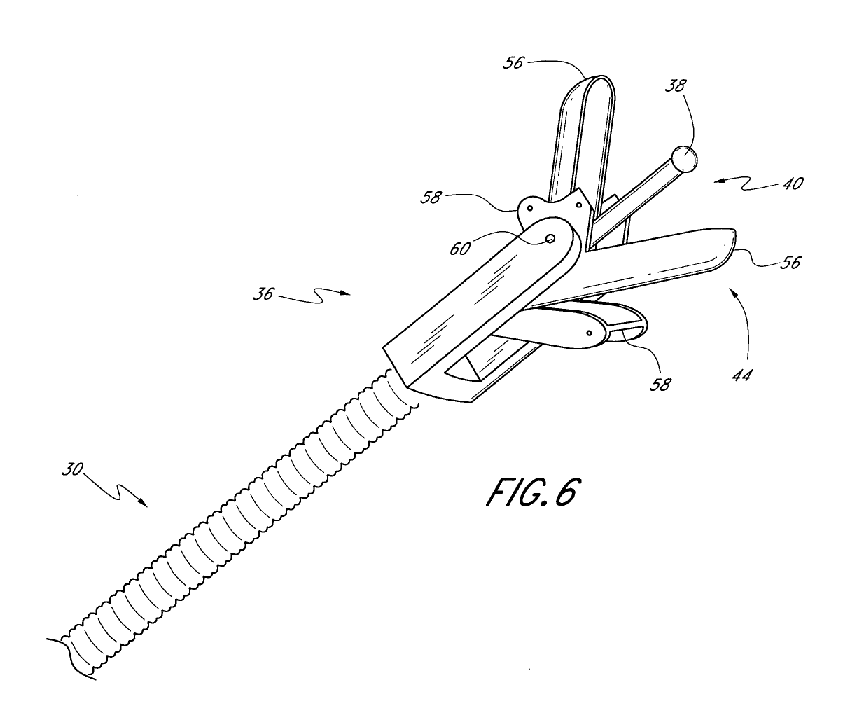

In a preferred embodiment, the caustic agent 38 is provided on an applicator tip 40 having a base 42, as illustrated in FIG. 4. The base 42 is configured to permit attachment thereof to the distal portion of the wire 30 via threads, clamps, clips, or other attachment means. FIG. 5A illustrates a schematic of the wire 30 with a base 42 for applying a caustic agent to a patient. The wire 30 is illustrated in a closed configuration, having a protective cover, such as a sheath 44 on a distal portion 46 thereof. As used herein, the term “protective sheath” is intended to have its ordinary meaning and is intended to mean, without limitation, a covering, shield, or other device that is configured to permit an enclosure or space therein.

The protective sheath 44 is configured to enclose, or cover, the therapeutic agent 38 and/or base 42 when the wire 30 is in the closed configuration. In some embodiments, the wire 30 has a lumen 50 extending from a proximal portion 48 to the distal portion 46. In one embodiment, an actuating wire 52 extends through the lumen 50 to actuate the protective sheath 44 on the distal portion 46 between the open and closed configuration. A button 54 is provided on the proximal portion 48, and the button 51 is connected to the actuating wire 52 for operation of the protective sheath 44.

Although the wire 30 is described as having a button 54 and an actuating wire 52, the wire 30 is not intended to be limited to the above description. For example, the button 54 may instead be a dial, switch, or other mechanical devices for actuating the actuating wire 52, or the button 54 may be an electronic device that is configured to actuate the actuating wire 52.

Additionally, the actuating wire 52 can be a tube extending through the wire 30 to provide mechanical operation of the protective sheath 44, or the actuating wire 52 may assume other configurations or constructions that permit transfer of a mechanical or electronic signal for actuating the protective sheaths. For example, an endoscopic treatment instrument that utilizes mechanical means is disclosed in U.S. Pat. No. 4,982,727, filed on Apr. 13, 1990, the entire contents of which are incorporated herein by reference and are made a part of this specification.

In some embodiments, the wire 30 is configured such that when the button 54 is depressed, the protective sheath 44 is opened, withdrawn, or otherwise removed from covering the caustic agent 38, such that the caustic agent 38 can be used on the tissue in the patient. FIG. 5B schematically illustrates the button 54 partially depressed and the protective sheath 44 partially open, thereby partially exposing the caustic agent 38 or base 42.

FIG. 5C schematically illustrates the button 54 fully depressed and the protective sheath 44 fully open to expose the caustic agent 38 for application. In one embodiment, the base 42 may be connected to the actuating wire 52 such that as the button 54 is depressed and the protective sheath 44 is withdrawn or opened, the base 42 is advanced distally in preparation for application.

FIG. 6 illustrates one embodiment of a protective sheath 44 that is configured to enclose the caustic agent 38 on the application tip 40. The distal tip 36 of the wire 30 includes two applicator shields 56 that are configured provide a hollow enclosure when in the closed configuration. In one embodiment, and as illustrated, the shields 56 may have a semi-hemispherical configuration. It is contemplated that the shields 56 may have other configurations. For example, the shields 56 may have polygonal or irregular shapes. The two shields 56 are configured to permit rotation thereof away from each other to expose the enclosure when in an open configuration. The shields 56 are connected to the actuating wire 52 for actuation between the open and closed configuration.

In some embodiments, each shield 56 includes a pivoting flange 58 that is positioned opposite the corresponding shield 56 such that the pivoting flange 58 is located on one side a pivoting joint 60, about which the shield 56 is configured to pivot. The pivoting flange 58 is coupled to the actuating wire 52 such that distal movement of the actuating wire 52 translates into distal movement of the pivoting flange 58. As the pivoting flange 58 is moved distally, the corresponding shield 56 is configured to rotate about the pivoting joint 60 to accommodate the movement of the flange 58. Accordingly, distal movement of the actuating wire 52 is translated into rotational movement of the shield 56 about the pivoting joint 60, thereby rotating the shield 56 to an open configuration.

In some embodiments, the pivoting flange 58 of each shield 56 may be configured to be actuated by withdrawal of a cable or wire in tension. For example, the pivoting flange 58 can be configured to be located on the same side of the pivoting joint 60 as the corresponding shield 56. The cable or wire can be configured to draw the pivoting flange 58 proximally when actuated. When the cable or wire draws the pivoting flange 58 proximally, the shield 56 pivots about the pivoting joint 60 to separate the shield 56 and expose the caustic agent 38.

As the shields 56 are moved or otherwise activated, into an open configuration, the caustic agent 38 is exposed for application on the patient's tissue. In some embodiments, the wire 30 may have more than one actuating wire 52 extending therethrough for actuation of the shields 56. It is contemplated that other means may be used to actuate the shields 56. For example, cables may be used to connect the button 34, or other actuating device, and the shields 56. It is contemplated that in some embodiments, the button 34, or other actuating device, may not be mechanically connected to the shields.

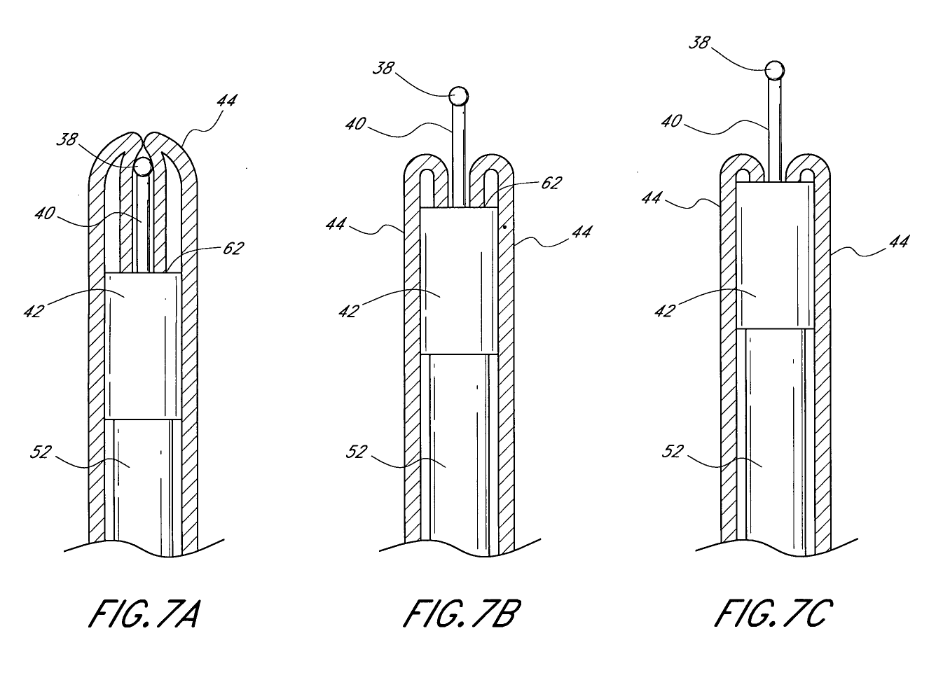

FIGS. 7A-C illustrate partial cross-sectional views of another embodiment of a protective sheath 44 configured to cover or enclose the caustic agent 38 before application of the agent 38 on tissue in the patient. In FIG. 7A, the caustic agent 38 is illustrated as substantially or fully enclosed by the protective sheath 44. The caustic agent 38 is positioned on an applicator tip 40 that extends distally from a base 42. The base 42 is preferably connected to the distal end of an actuating rod or wire 52. A proximal end (not shown) of the actuating wire 52 is connected to the button 34, 54, or other actuating device. The wire 52 is configured for axial movement when actuated by the button 34 or other actuating device.

The protective sheath 44 is preferably connected to a portion of the base 42, applicator tip 40, or actuating wire 52 at a distal end 62 of the sheath 44. When in the closed configuration, the protective sheath 44 extends distally to a distal-most portion, whereupon the protective sheath 44 unravels and thereafter extends proximally. The proximal end (not shown) of the protective sheath 44 is preferably connected to the button 34, or other actuating device, that is configured to move the sheath 44 axially with respect to the base 42.

In some embodiments, the button 34 is connected to the proximal end (not shown) of the actuating wire 52, and the proximal end (not shown) of the protective sheath 44 is connected to a button housing 64, which encases the button 34. The sheath 44 extends distally, fully encasing the actuating wire 52 in the lumen of the sheath 44, to a distal-most point of the sheath 44, whereupon the sheath 44 is folded inward and extends proximally within itself to connect to the base 42 at the distal end 62. Accordingly, when the button 34 is depressed, the button 34 advances the actuating wire 52 distally while the button housing 64 and the sheath 44 are drawn proximally. As the base 42 is advanced distally and the sheath 44 is withdrawn proximally, the distal-most portion of the sheath 44 unravels to expose the caustic agent 38, as illustrated in FIGS. 7A-C.

In FIG. 7B, the sheath 44 is shown as partially withdrawn to expose a portion of the applicator tip 40 and the caustic agent 38. FIG. 7C illustrates a configuration in which the sheath 44 is fully withdrawn, and the caustic agent 38 is prepared for application. Although the figures depict the sheath 44 connected at its distal end to the base 42, other configurations may also be used for connecting the sheath 44 to the actuating wire 52. For example, the sheath 44 may be connected directly to the actuating wire 52.

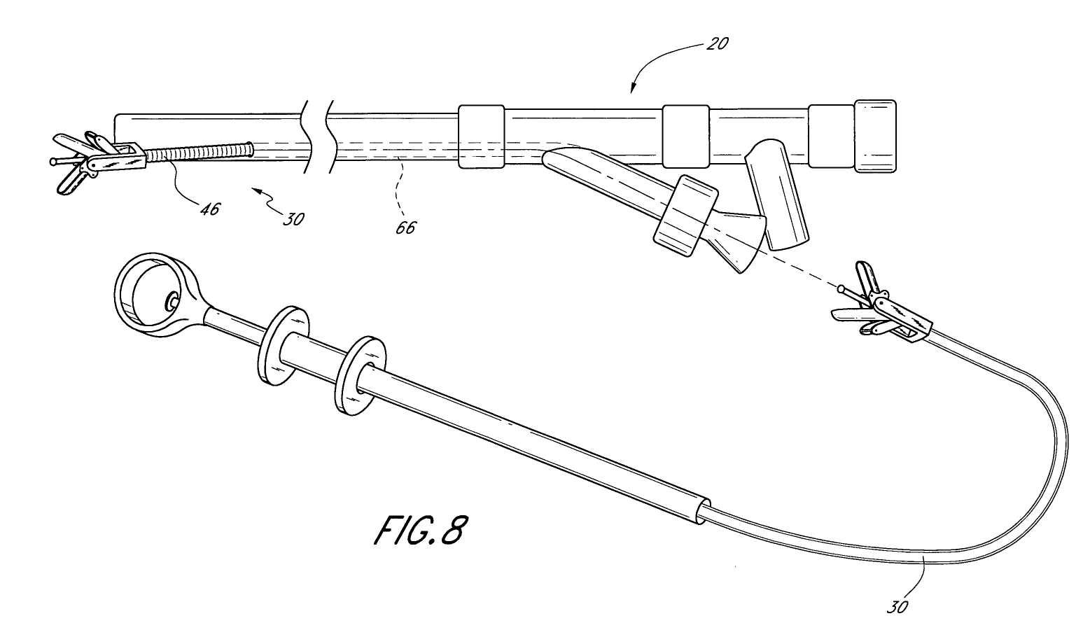

FIG. 8 depicts a rigid endoscope 20 having a lumen 66 extending therethrough. The lumen 66 is configured to permit placement therethrough of the operating wire 30 such that the distal portion 46 of the wire 30 is able to be advanced distal of the distal end of the endoscope 20. FIG. 9 depicts another embodiment of a rigid endoscope 20 that is configured to permit placement therethrough of a rigid wire 30. The rigid wire 30 depicted may operate as disclosed with respect to various embodiments disclosed herein. For example, the illustrated rigid wire 30 is depicted with a button 54 and a protective sheath 44 on the distal portion 46 of the wire 30.

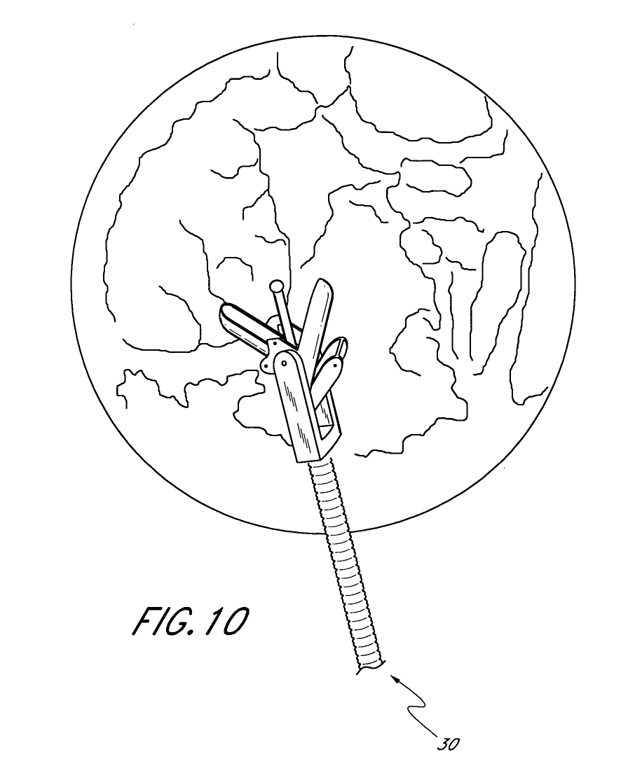

In one mode of operation, the endoscope 20 is advanced into the patient to a predetermined or otherwise desired position. The endoscope 20 is used by the practitioner to view or image the structure or lesion to be diagnosed or treated. The wire 30 is advanced through a lumen 66 extending through the endoscope 20 for operating tools, such that a distal portion of the wire 30 extends distal of the endoscope 20. In some embodiments, as depicted in FIG. 10, the operating wire 30 is advanced within the line of sight of the endoscope 20 such that the wire 30 may be viewed by the practitioner during application of the caustic agent 38.

The distal portion 46 of the wire 30 preferably encloses, conceals, or otherwise encapsulates the caustic agent 38 while being advanced into position to reduce the likelihood of inadvertent or undesirable application of the agent 38. The wire 30 is then actuated or prepared for deployment or application of the caustic agent 38 on the desired tissue.

In some embodiments, the protective sheath 44 is removed or opened from covering the caustic agent 38 such that the agent is exposed, uncovered, revealed, or otherwise prepared for deployment. The wire 30 is then advanced as desired by the practitioner to apply the caustic agent 38 to the tissue. Upon completion of the application, the wire 30 may re-sheath, enclose, or cover the caustic agent 38 to reduce the likelihood of inadvertent or undesirable application of the agent 38 during withdrawal thereof. The wire 30 can be withdrawn through the lumen 66 of the endoscope 20.

In some applications, multiple applications of the caustic agent 38 may be desirable. In such applications, the endoscope 20 can be configured to permit repeated application of wires 30 through the lumen 66 for application. For example, following the procedure described above, the endoscope 20 may be advanced or positioned to a second predetermined or desired position for application of the caustic agent 38. A second wire 30 may be inserted following removal of the first wire 30, and the above-described may be repeated for application of the caustic agent 38 at the second location. This process may be repeated as necessary.

In some embodiments, the applicator tip 40 or base 42 may be configured for removal from the actuating wire 52. Accordingly, once the first application of the caustic agent 38 is applied, the wire 30 may be removed, and the used portion of the wire 30 may be replace with a new portion. For example, the used portion can be a removable base 42 that is connected to the actuating wire 52 by threads, clamps, or other detachable means. When the used base 42 is withdrawn from the scope 20 with the wire 30, a new base 42 may be detachably connected to the wire 30, and the same wire 30 can be used in the subsequent treatment.

In some embodiments, both the endoscope 20 and the wire 30 may be controllable within the patient. For example, U.S. Pat. No. 5,908,381, filed Apr. 30, 1997, the entire contents of which are hereby incorporated by reference and made a part of this specification, describes further embodiments in which a controllable wire 30 is extended from the scope 20 for diagnostic or therapeutic treatment of a patient.

The practitioner may use the above devices and methods for achieving hemostasis with a patient suffering from epistaxis by the application of a caustic agent. Additionally, the practitioner may use the devices and methods for application of a caustic agent, such as silver nitrate, to granulations or other growths within a patient's airways. While the above devices and methods are described for application of a caustic agent, it is contemplated that the principles of the present application may be applicable to the application of other agents. For example, other diagnostic or therapeutic agents may be configured for placement on the applicator tip and may be applied in a similar manner as described above with reference to the caustic agent. For example, other therapeutic treatments for the airways of a patient are disclosed in U.S. Pat. No. 6,846,799, filed Jul. 14, 2000, and U.S. Pat. No. 6,846,486, filed Feb. 24, 2000, the entireties of both of which are hereby incorporated by reference and made a part of this specification.

Although the present disclosure has been provided in the context of certain preferred embodiments and examples, it will be understood by those skilled in the art that the present disclosure extends beyond the specifically disclosed embodiments to other alternative embodiments and/or uses of the present disclosure and obvious modifications and equivalents thereof. In addition, while a number of variations of the present disclosure have been shown and described in detail, other modifications, which are within the scope of the present disclosure, will be readily apparent to those of skill in the art based upon this disclosure. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein provided should not be limited by the particular disclosed embodiments described above.

What is claimed is:

1. An endoscopic device comprising:
a protective cover configured to be positionable in an open configuration and a closed configuration, the protective cover being sized and shaped for insertion through a lumen in an endoscope;
an applicator tip configured to apply a therapeutic agent to a tissue, the applicator tip being configured to be at least partially covered by the protective cover when the protective cover is in a closed configuration, the applicator tip further being configured to be exposed when the protective cover is in an open configuration; and
an actuator that is configured to move the protective cover between the open configuration and the closed configuration.

2. The endoscopic device of claim 1, wherein the therapeutic agent comprises a caustic agent.

3. The endoscopic device of claim 2, wherein the caustic agent comprises silver nitrate.

4. The endoscopic device of claim 1, wherein the protective cover comprises a plurality of semi-hemispherical shields.

5. The endoscopic device of claim 4, wherein at least one of the plurality of shields comprise a hollow enclosure that is configured to accommodate a portion of the applicator tip.

6. The endoscopic device of claim 1, wherein the device is configured for insertion through a lumen of an endoscope.

7. The endoscopic device of claim 1, wherein the actuator comprises a button that is configured to move the protective cover between the open configuration and the closed configuration when the button is depressed.

8. An endoscopic therapeutic device comprising:
a endoscope configured for viewing of a patient, the endoscope having a lumen therethrough for insertion of an operating instrument; and
an operating member having a proximal end and a distal end, the distal end of the operating member comprising a protective covering that is configured to enclose therein a therapeutic agent, the proximal end of the operating member being configured to be connected to an actuator, the actuator being configured to activate movement of the protective covering and expose the caustic agent, the operating member being configured for insertion through the lumen of the endoscope.

9. The endoscopic device of claim 8, wherein the therapeutic agent comprises a caustic agent.

10. The endoscopic therapeutic device of claim 8, wherein the protective covering comprises a plurality of shields.

11. The endoscopic therapeutic device of claim 8, wherein the protective covering comprises a sheath that extends from a proximal portion of an actuating wire that extends through the operating member to a distal portion of the actuating wire.

12. The endoscopic therapeutic device of claim 11, wherein the sheath is configured to be axially moveable with respect to the actuating wire.

13. The endoscopic therapeutic device of claim 8, wherein the caustic agent comprises silver nitrate.

14. A method for treating a lesion in an airway of a patient, the method comprising:
positioning an endoscope in an airway of a patient at a location proximate to a lesion;
inserting through a lumen of the endoscope an operating member that comprises a distal end that is configured to contain a caustic agent;
advancing the operating member to a position where a distal portion of the operating member extends from the endoscope and is adjacent the lesion;
actuating the operating member to expose the caustic agent;
applying the caustic agent to the lesion.

15. The method of claim 14, further comprising actuating the operating member to cease exposing the caustic agent.

16. The method of claim 14, wherein the application of the caustic agent is viewed through the endoscope.

17. A method for treating epistaxis in a patient, the method comprising:
positioning an endoscope in the patient at a predetermined or desired location adjacent the hemorrhage;
inserting through a lumen of the endoscope an operating member that comprising a distal end that is configured to conceal a caustic agent;
advancing the operating member to a position where a distal portion of the operating member extends from the endoscope and is adjacent the hemorrhage;
actuating the operating member to expose the caustic agent;
applying the caustic agent to the hemorrhage; and
actuating the operating member to conceal the caustic agent.

18. The method of claim 17, wherein actuation of the operating member is achieved by an actuation button on a distal portion of the operating member.

19. An endoscopic therapeutic device comprising:
a protective cover having a proximal end and a distal end, the proximal end being connected to a first portion of an actuator; and
an applicator comprising a therapeutic agent located on a distal end thereof, the distal end of the protective cover being connected to the applicator, and the protective cover covering a portion of the applicator in a closed configuration, the proximal end of the applicator being connected to a second portion of the actuator;
wherein the protective cover is configured for axial movement with respect to the applicator when the actuator is operated to change the configuration of the device between a closed configuration and an open configuration.

20. The endoscopic therapeutic device of claim 19, wherein the therapeutic agent comprises a caustic agent.

21. The endoscopic therapeutic device of claim 20, wherein the caustic agent comprises silver nitrate.

22. The endoscopic therapeutic device of claim 19, wherein the protective cover unravels to expose the applicator comprising a therapeutic agent when in an open configuration.
